(19) United States
(12) Patent Application Publication
(10) Pub. No.: US 2004/0038295 A1
(43) Pub. Date: Feb. 26, 2004

(54) Assays

(76) Inventors: Thomas William Rademacher, Oxford (GB); Stephanie Bord, Marseille (FR)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/276,168

(22) PCT Filed: May 11, 2001
(86) PCT No.: PCT/GB01/02082

(30) Foreign Application Priority Data
May 12, 2000 (GB) 0011590.7
Feb. 1, 2001 (GB) 0102566.7

(51) Int. Cl. G01N 33/53

(52) U.S. Cl. 435/7.1

(57) ABSTRACT

Assays, kits and methods for determining the presence or amount inositol phosphoglycans (IPG) analytes in samples are disclosed based on the finding that IPG antigens are capable of binding to gelatin. These assays can be used in the diagnosis of conditions where the presence or amount of these analytes is a diagnostic marker for a condition. Methods for the diagnosis of pre-eclampsia, distinguishing different type of pre-eclampsia, are disclosed and also methods for determining the onset of labour in a patient.

### FIELD OF THE INVENTION

The present invention relates to materials and methods for use in assays for determining the presence or amount of an IPG analyte in a sample, and in particular for the diagnosis of pre-eclampsia, distinguishing different types of pre-eclampsia and predicting the onset of labour.

### BACKGROUND OF THE INVENTION

Many of the actions of growth factors on cells are thought to be mediated by a family of inositol phosphoglycan (IPG) second messengers (Rademacher et al, 1994). It is thought that the source of IPGs is a “free” form of glycosyl phosphatidylinositol (GPI) situated in cell membranes. IPGs are thought to be released by the action of phosphatidylinositol-specific phospholipases following ligation of growth factors to receptors on the cell surface. There is evidence that IPGs mediate the action of a large number of growth factors including insulin, nerve growth factor, hepatocyte growth factor, insulin-like growth factor I (IGF-I), fibroblast growth factor, transforming growth factor β, the action of IL-2 on B-cells and T-cells, ACTH signalling of adrenocortical cells, IgE, FSH and hCG stimulation of granulosa cells, thyrotropin stimulation of thyroid cells, cell proliferation in the early developing ear and rat mammary gland.

Soluble IPG fractions have been obtained from a variety of animal tissues including rat tissues (liver, kidney, muscle brain, adipose, heart) and bovine liver. IPG biological activity has also been detected in malaria parasitized red blood cells (RBC) and mycobacteria. We have divided the family of IPG second messengers into distinct A and P-type subfamilies on the basis of their biological activities. In the rat, release of the A and P-type mediators has been shown to be tissue-specific (Kunjara et al, 1995).

WO98/10791 discloses that members of the P-type IPG family are a diagnostic marker for pre-eclampsia. This is an important observation because pre-eclampsia is a potentially fatal condition affecting up to 10% of all pregnancies, causing maternal endothelial dysfunction and problems with activation of the clotting system, increased vascular permeability and ischaemia in maternal organs secondary to vasoconstriction.

WO98/11435 further discloses that the ratio of P and A-type IPGs can be used in the diagnosis of diabetes, and in particular type II diabetes.

WO99/00844 describes the production of monoclonal and polyclonal antibodies capable of binding to IPGs, and their use in diagnostic assays as binding agents for capturing IPG antigens in samples and as labelled developing agents for determining the presence or amount of the IPG antigens. WO99/00844 exemplifies the sandwich ELISA assays using a monoclonal capture antibody, a developing antibody capable of binding to bound IPG antigens and an enzyme labelled, polyclonal detection antibody. These assays are disclosed as being useful in the diagnosis of pre-eclampsia, type II diabetes and in the diagnosis of a susceptibility to type I diabetes.

However, despite these advances in the art, there is a continuing need in the art for assays which assist in the diagnosis of these conditions and in particular for a simple, easy to use assays for home use.

### SUMMARY OF THE INVENTION

Broadly, in some aspects, the present invention concerns materials and methods for assays for determining the presence or amount of inositol phosphoglycans (IPG) antigens in a sample, based on the finding that certain IPG antigens are capable of binding to gelatin. These assays can be used in the diagnosis of conditions where the presence or amount of these analytes is a diagnostic marker for a condition, such as pre-eclampsia, even distinguishing different type of pre-eclampsia. In other aspects, the present invention relates to new findings concerning the correlation between IPG levels and the onset of labour in a patient. This test can be carried out employing the new assays disclosed herein or those described in the prior art.

We have previously disclosed in WO98/10791 the correlation between elevated levels of IPGs, and in particular P-type IPGs, and the occurrence of pre-eclampsia. In one embodiment disclosed herein, we now show that the pre-eclamptic urinary antigen which is a diagnostic-marker-for pre-eclampsia can be captured in an assay using a gelatin capture phase. These observations open up the possibility of making simple assay devices for determining the presence or amount of the IPG analytes present in samples.

Accordingly, in a first aspect, the present invention provides the use of gelatin as a binding agent in an assay for determining the presence or amount of an inositol phosphoglycan (IPG) analyte in a sample.

In a further aspect, the present invention provides a method of diagnosing a condition associated with the presence or amount of an inositol phosphoglycan (IPG) analyte in a sample from a patient, the method comprising:

contacting the sample with a solid support having a capture zone comprising gelatin which is capable of binding the IPG analyte present in the sample;

contacting the solid support with a developing agent capable of binding to the captured IPG analyte; and,

detecting the developing agent to determine the presence or amount of the IPG analyte in the sample.

In a further aspect, the present invention provides a kit for diagnosing a condition associated with the presence or amount of an inositol phosphoglycan (IPG) analyte in a sample from a patient, the kit comprising:

a solid support having a capture zone comprising gelatin which is capable of binding to the IPG analyte present in the sample;

a developing agent capable of binding to the IPG analyte bound to the capture zone, wherein the developing agent comprises a detectable label, a moiety capable of being converted into a detectable label or is capable of specifically interacting with a further detectably labelled reagent.

In a further aspect, the present invention provides a lateral flow device for determining the presence or amount of an inositol phosphoglycan (IPG) analyte in a sample, the device comprising a solid support comprising in sample flow order:

(a) a sample addition zone;

(b) a pre-treatment zone for reacting with the sample;

(c) a capture zone comprising gelatin which is capable of binding to the IPG analyte present in the sample;

wherein the presence of amount of the IPG analyte is determined using a developing agent capable of binding to the IPG analyte bound to the capture zone, the developing agent comprises a detectable label, a moiety capable of being converted into a detectable label or is capable of specifically interacting with a further detectably labelled reagent. In this aspect of the invention, preferably the pre-treatment zone adjusts the pH of the sample to enhance the binding of the IPG analyte to the gelatin capture phase.

The observations underlying the present invention were made in the development of assays for IPG analytes. In these experiments, we surprisingly found that the IPG analytes were capable of binding to gelatin used as a blocking agent in sandwich assays (i.e. using anti-IPG antibodies as capture and developing agents). These experiments showed that gelatin was capable of capturing the IPG analytes present in test samples.

In a further aspect, the present invention relates to further refinements to assays and methods for the diagnosis of pre-eclampsia. As reported in the examples below, by measuring the level or amount of IPGs in a sample, and in particular P-type IPGs, it is possible to define two types of pre-eclampsia in pregnant patients. In a first type, the assays and methods described herein are predictive of the development of pre-eclampsia at least 2 weeks, more preferably at least 3 weeks and most preferably at least 4 weeks before the manifestation of clinical symptoms. Thus, the present invention is particularly advantageous for the diagnosis and clinical management of such patients as treatment can begin well before the development of pre-eclamptic complications. In the second class of patients, although the assay is not predictive, it is a useful diagnostic for pre-eclampsia, with a positive result in the assay strongly correlating with the development of pre-eclampsia. In both cases, the assays and method disclosed herein provide results which correlate with the severity of pre-eclampsia, providing further useful information for the diagnosis and prognosis of this condition.

Accordingly, in a further aspect, the present invention provides the use of the level or amount of P-type IPGs for diagnosing pre-eclampsia prior to the onset of its clinical symptoms.

In a further aspect, the present invention concerns the further finding that the onset of labour correlates with the level, and more especially an elevated level, of P-type IPGs in pregnant female mammals. Thus, the present invention provides a method of predicting the onset of labour in a female mammal, the method comprising determining the amount of P-type IPGs and/or the activity of P-type IPGs in a sample from the mammal. Thus, a determination of the likely time of onset of labour can then be made by correlating the result of this assay with corresponding amounts or activities of P-type IPGs from control, e.g. value from known labouring and non-labouring groups. The results described herein show that the change in the level of P-type IPGs rises dramatically before the onset of labour, rising 5-fold over the pre-labour levels. In addition, it may be possible to use this indicator to distinguish between pre-term and normal term labour. The test disclosed herein can be used in both the medical and veterinary fields. In the latter case, a test to detect the onset of labour would be of considerable use, e.g. in lambing.

In one embodiment, the method comprises the steps of:

(a) contacting a biological sample obtained from the patient with a solid support having immobilised thereon binding agent having binding sites specific for one or more P-type IPGs;

(b) contacting the solid support with a labelled developing agent capable of binding to unoccupied binding sites, bound P-type IPGs or occupied binding sites; and,

(c) detecting the label of the developing agent specifically binding in step (b) to obtain a value representative of the amount or activity of the P-type IPGs in the sample.

As set out below, in this aspect of the invention, the amount or activity of the P-type IPGs can be further confirmed using a marker which correlates with the level of the P-type IPGs.

Embodiments of the present invention will now be described by way of example and not limitation with reference to the accompanying figures.

### BRIEF DESCRIPTION OF THE DRAWINGS

FIG. 1 shows the effect of the concentration of the 2D1 monoclonal capture antibody with different pre-eclamptic urine samples.

FIG. 2 shows assays carried out at different dilutions of pre-eclamptic urine samples with and without the 2D1 capture antibody.

FIG. 3 shows the ability of different gelatin based reagents in binding IPG analytes.

FIG. 4 shows the effect of heat as a pretreatment method for pre-eclamptic urine samples.

FIG. 5 shows the effect of acid and heat as a pretreatment method for pre-eclamptic urine samples.

FIG. 6 shows the effect of HCl treatment on the binding of the IPG analyte in pre-eclamptic urine and how this varies with pH.

FIG. 7 shows a graph of control pregnant (n=47) and pre-eclamptic (n=27) urines tested using a gelatin capture phase assay.

FIG. 8 shows the signal development over time in examples of patients having pre-eclampsia where the assay is predictive of the development of pre-eclampsia and diagnostic of pre-eclampsia.

FIG. 9 shows the correlation between IPG levels and the day on which the labour began in urine samples taken on different days from patients having non pre-eclamptic pregnancies.

### DETAILED DESCRIPTION

IPG Analytes

WO98/10791 discloses the correlation between the over production or elevated bioactivity of IPGs, and in particular P-type IPG family members, and the occurrence of pre-eclampsia. WO98/11435 discloses the use of the amount or ratio of P and A-type IPGs in the diagnosis of diabetes. The further work described herein now shows that IPG analytes, such as the pre-eclamptic urinary antigen, can be captured using a gelatin capture phase.

In the present invention, “IPG analyte” includes an IPG or IPG family member, or a derivative, a precursor, a biosynthetic derivative or modified form thereof, the IPG analyte having the property of binding to a gelatin capture phase, as can be determined by the skilled person using the assays described herein. Preferably, the IPG analyte includes a lipid group as this is believed to improve the binding of the IPG analyte to the gelatin capture phase. The IPG analyte may be an IPG having these properties or a glycosyl phosphatidyl inositol (GPI), an IPG precursor including one or more lipid groups. An example of IPG analytes include the pre-eclamptic urinary antigen described herein.

The sample suspected on containing one or more IPG analytes of interest can be obtained from an appropriate source. In the case of biological materials, a sample of a body fluid such as urine, blood, serum, plasma, saliva, tears or mucus can be obtained from a patient for use in the assay. The use of urine samples for the diagnosis of pre-eclampsia is preferred.

The sample may be subjected to one or pre-treatment steps prior to carrying out the assay, for example to remove one or more biological contaminants or to treat the IPG analyte to make it more reactive to the capture zone, e.g. by heating to 90° C., cooling to −20° C. or by chemically treating the analyte. A preferred chemical treatment employs acid, e.g. HClO₄, TCA or especially HCl. Alternatively, alkali can be used, e.g. 100 mM NaOH. In embodiments in which a change in pH is used as a pre-treatment step, preferably the pH of the sample is adjusted to between about pH 0.0 to 2.5, and more preferably between about pH 0.5 to 2.5. In a preferred embodiment, the pre-treatment step employs a pH of about 1.0, e.g. as obtained using 100 mM HCl. Without wishing to be bound by any particular theory, it is believed that IPG antigens comprising lipid groups are particularly reactive with the gelatin in the capture zone, and the pre-treatment step assists in the formation of micelles of IPG analyte which readily bind to the gelatin without the need for a specific capture agent such as an antibody.

Studies have shown that A-type mediators modulate the activity of a number of insulin-dependent enzymes such as cAMP dependent protein kinase (inhibits), adenylate cyclase (inhibits) and cAMP phospho-diesterases (stimulates). In contrast, P-type mediators modulate the activity of insulin-dependent enzymes such as pyruvate dehydrogenase phosphatase (stimulates), glycogen synthase phosphatase (stimulates) and cAMP dependent protein kinase (inhibits). The A-type mediators mimic the lipogenic activity of insulin on adipocytes, whereas the P-type mediators mimic the glycogenic activity of insulin on muscle. Both A and P-type mediators are mitogenic when added to fibroblasts in serum free media. The ability of the mediators to stimulate fibroblast proliferation is enhanced if the cells are transfected with the EGF-receptor. A-type mediators can stimulate cell proliferation in chick cochleovestibular ganglia.

Soluble IPG fractions having A-type and P-type activity have been obtained from a variety of animal tissues including rat tissues (liver, kidney, muscle brain, adipose, heart) and bovine liver. A and P-type IPG biological activity has also been detected in human liver and placenta, malaria parasitized RBC and mycobacteria. The ability of a polyclonal cross-reacting anti-inositolglycan antibody to inhibit insulin action on human placental cytotrophoblasts and BC3H1 myocytes or bovine-derived IPG action on rat diaphragm and chick cochleovestibular ganglia suggests cross-species conservation of many structural features. However, it is important to note that although the prior art includes these reports of A and P-type IPG activity in some biological fractions, the purification or characterisation of the agents responsible for the activity was not disclosed until it was reported in the references below.

A-type substances are cyclitol-containing carbohydrates, also containing Zn²⁺ ion and optionally phosphate and having the properties of regulating lipogenic activity and inhibiting cAMP dependent protein kinase. They may also inhibit adenylate cyclase, be mitogenic when added to EGF-transfected fibroblasts in serum free medium, and stimulate lipogenesis in adipocytes.

P-type substances are cyclitol-containing carbohydrates, also containing Mn²⁺ and/or Zn²⁺ ions and optionally phosphate and having the properties of regulating glycogen metabolism and activating pyruvate dehydrogenase phosphatase. They may also stimulate the activity of glycogen synthase phosphatase, be mitogenic when added to fibroblasts in serum free medium, and stimulate pyruvate dehydrogenase phosphatase.

Methods for obtaining A-type and P-type IPGs are set out in detail in Caro et al, 1997, and in WO98/11116 and WO98/11117. Methods for obtaining the free GPI precursors of the A and P-type IPGs are set out below.

Solid Supports

A wide variety of materials that can be used to produce the solid support are known in the art including glass, plastics supports such as (a) polystyrene or nylon and copolymers and mixtures thereof, (b) microspheres made from polystyrene, latex or other materials and (c) lateral flow solid supports such as dipsticks or printed liquidic circuits (see EP 0 590 695 A, GB 2 261 283 A and GB 2 261 284 A).

For simple dipstick assay formats, the solid phase may be a cellulose ester, and materials such as nitrocellulose are preferred. It should be understood that the term “nitrocellulose” refers to nitric acid esters of cellulose which may be nitrocellulose alone, or a mixed ester of nitric acid and other acids, in particular, aliphatic carboxylic acids having from one to seven carbon atoms, with acetic acid being preferred. Such solid supports which are formed from cellulose esterified with nitric acid alone or a mixture of nitric acid and another acid such as acetic acid, are often referred to as nitrocellulose paper.

In some embodiments, the solid support provides a surface on which the gelatin which acts as binding agent or capture zone for the IPG analyte can be coated or otherwise immobilised in a location on the solid support. The solid support employed in the assay is preferably in sheet form, with the substrate in sheet form, generally being in the form of a card, a test strip or dipstick. Alternatively, the solid support may be largely composed of gelatin. In some embodiments, the solid support may have predefined capture zones so that a plurality of analytes can be simultaneously or sequentially tested using a single solid support. The use of dipstick assays is very well known in the art and these known assays could readily be adapted for use in the present invention, e.g. by substituting a binding agent such as an antibody for the gelatin capture phase of the invention.

Gelatin is a complex glycoprotein typically obtained from boiling animal cartilage or collagen in water. A variety of different types of gelatin are known in the art and are suitable for use in the present invention, including Boehringer Mannheim's proprietary gelatin blocker, Pierce Superblock and Sigma gelatin hydrolysate or similar agents which are all capable of binding to lipidic IPGs.

Developing Agents

In the assays described herein, the presence or amount of an IPG analyte on the gelatin capture phase can be determined by using a developing agent which binds to the IPG analyte and/or by determining a biological activity of the IPG analyte as a measure of the amount of analyte captured. Examples of the biological activities of IPGs are provided above.

In preferred embodiments, the presence or amount of the IPG analyte bound to the capture zone/binding agent can be determined using a developing agent which is capable of binding to the IPG analyte. The developing agent binds to captured IPG analyte and is detected to provide the result of the assay. The detection of the developing agent can be carried out using a detectable label, a moiety capable of being converted into a detectable label or a moiety capable of interacting specifically with a further detectably labelled reagent.

Generally, the developing agent is typically tagged with a label or reporter molecule which can directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. Any method known in the art for separately conjugating the label or reporter molecule to a developing agent which is a polypeptide (e.g. an anti-IPG antibody) may be employed, including those methods described by Hunter et al, Nature 144:945, 1962; David et al, Biochemistry 13:1014, 1974; Pain et al, J. Immunol. Meth. 40:219, 1981; and Nygren, J. Histochem. and Cytochem. 30:407, 1982.

The use of a wide range of labels is well known to those skilled in the art. Preferred label for simple assays are gold particles or enzyme labels, e.g. for use in ELISA type assays. In these assays, the developing agent is or can be conjugated to an enzyme. In the latter case, a developing agent such as an anti-IPG antibody can be used to bind to the captured IPG analyte and then detected using an anti-species enzyme labelled antibody. After the binding reactions between the capture zone and the analyte have taken place, the result of the assay is obtained by contacting the enzyme with a substrate on which acts to produce an observable result such as a colour change, the extent of which depends on the presence or amount of analyte originally in the sample. Other refinements using gold labelling include the use of silver enhanced gold labelling (SEGLISA), e.g. as disclosed in WO91/01003.

One favoured mode is by covalent linkage of each developing agent with an individual fluorochrome, phosphor or laser dye with spectrally isolated absorption or emission characteristics. Suitable fluorochromes include fluorescein, rhodamine, luciferin, phycoerythrin and Texas Red. Suitable chromogenic dyes include diaminobenzidine. Other detectable labels include radioactive isotopic labels, such as ³H, ¹⁴C, ³²P, ³⁵S, ¹²⁶I, or ^{99m} Tc, and enzyme labels such as alkaline phosphatase, β-galactosidase or horseradish peroxidase, which catalyze reactions leading to detectable reaction products and can provide amplification of signal.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyze reactions that develop or change colours or cause changes in electrical properties, for example. They may be molecularly excitable, such that electronic transitions between energy states result in characteristic spectral absorptions or emissions. They may include chemical entities used in conjunction with biosensors.

Methods of detecting such labels are well known in the art. By way of example, radioactive labels can be detected using a scintillation counter or other radiation counting device, fluorescent labels using a laser and confocal microscope, and enzyme labels by the action of an enzyme label on a substrate, typically to produce a colour change.

Conveniently, the developing agent binds to the IPG analyte as it comprises a specific binding member for the analyte, in the sense that it binds to the IPG analyte in preference to other substances and in particular, other substances which may be present in the sample. In a preferred embodiment, the developing agent is an anti-IPG antibody, e.g. monoclonal antibody 2D1 or 5H6, deposited at ECACC under accession numbers 98031212 or 98030901 respectively.

The production of monoclonal and polyclonal antibodies capable of specifically binding to P and A-type IPGs are disclosed in WO99/00844. These antibodies can be used in the assays disclosed in this application, optionally being modified using techniques which are standard in the art. Antibodies similar to those exemplified for the first time here can also be produced using the teaching herein in conjunction with known methods. These methods of producing antibodies include immunising a mammal (e.g. mouse, rat, rabbit, horse, goat, sheep or monkey) with the IPG or a GPI, or fragments of these molecules. Antibodies may be obtained from immunised animals using any of a variety of techniques known in the art, and screened, preferably using binding of antibody to antigen of interest. Isolation of antibodies and/or antibody-producing cells from an animal may be accompanied by a step of sacrificing the animal.

As an alternative or supplement to immunising a mammal with an IPG, an antibody specific for an IPG may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the IPGs (or fragments, derivatives or biosynthetic intermediates), or may be one constructed using sequences obtained from an organism which has been exposed to the antigen of interest.

The term “monoclonal antibody” as used herein refers to an antibody obtained from a substantially homogenous population of antibodies, i.e. the individual antibodies comprising the population are identical apart from possible naturally occurring mutations that may be present in minor amounts. Monoclonal antibodies can be produced by the method first described by Kohler and Milstein, Nature, 256:495, 1975 or may be made by recombinant methods, see Cabilly et al, U.S. Pat. No. 4,816,567, or Mage and Lamoyi in Monoclonal Antibody Production Techniques and Applications, pages 79-97, Marcel Dekker Inc, New York, 0.1987.

In the hybridoma method, a mouse or other appropriate host animal is immunised with the antigen by subcutaneous, intraperitoneal, or intramuscular routes to elicit lymphocytes that produce or are capable of producing antibodies that will specifically bind to the IPG-used for immunisation. Alternatively, lymphocytes may be immunised in vitro. Lymphocytes then are fused with myeloma cells using a suitable fusing agent, such as polyethylene glycol, to form a hybridoma cell, see Goding, Monoclonal Antibodies: Principles and Practice, pp. 59-103 (Academic Press, 1986). Immunisation with soluble IPG or GPI and via the intraperitoneal route shown in the examples was surprisingly effective in producing antibodies specific for IPGs.

The hybridoma cells thus prepared can be seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody producing cells, and are sensitive to a medium such as HAT medium.

Culture medium in which hybridoma cells are growing is assayed for production of monoclonal antibodies directed against the IPGs. Preferably, the binding specificity is determined by enzyme-linked immunoabsorbance assay (ELISA). The monoclonal antibodies of the invention are those that specifically bind to either or both P and A-type IPGs.

Preferably, an antibody based developing agent will have an affinity which is greater than micromolar or greater affinity (i.e. an affinity greater than 10⁻⁶ mol) as determined, for example, by Scatchard analysis, see Munson & Pollard, Anal. Biochem., 107:220, 1980.

After hybridoma cells are identified that produce neutralising antibodies of the desired specificity and affinity, the clones can be subcloned by limiting dilution procedures and grown by standard methods. Suitable culture media for this purpose include Dulbecco's Modified Eagle's Medium or RPM1-1640 medium. In addition, the hybridoma cells may be grown in vivo as ascites tumours in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Nucleic acid encoding the monoclonal antibodies of the invention is readily isolated and sequenced using procedures well known in the art, e.g. by using oligonucleotide probes that are capable of binding specifically to genes encoding the heavy and light chains of murine antibodies. The hybridoma cells of the invention are a preferred source of nucleic acid encoding the antibodies or fragments thereof. Once isolated, the nucleic acid is ligated into expression or cloning vectors, which are then transfected into host cells, which can be cultured so that the monoclonal antibodies are produced in the recombinant host cell culture.

Hybridomas capable of producing antibody with desired binding characteristics are within the scope of the present invention, as are host cells containing nucleic acid encoding antibodies (including antibody fragments) and capable of their expression. The invention also provides methods of production of the antibodies including growing a cell capable of producing the antibody under conditions in which the antibody is produced, and preferably secreted.

Antibodies according to the present invention may be modified in a number of ways. Indeed the term “antibody” should be construed as covering any binding substance having a binding domain with the required specificity. Thus, the invention covers antibody fragments, derivatives, functional equivalents and homologues of antibodies, including synthetic molecules and molecules whose shape mimics that of an antibody enabling it to bind an antigen or epitope, here an IPG analyte.

Examples of antibody fragments, capable of binding an antigen or other binding partner, are the Fab fragment consisting of the VL, VH, CL and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)₂ fragments, a bivalent fragment including two Fab fragments linked by a disulphide bridge at the hinge region. Single chain Fv fragments are also included.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies, humanised antibodies or chimeric molecules which retain the specificity of the original antibody. Such techniques may involve, introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2 188 638 A or EP 0 239 400 A. Cloning and expression of chimeric antibodies are described in EP 0 120 694 A and EP 0 125 023 A.

Diagnostic Methods

Methods for determining the concentration of analytes in biological samples from individuals are well known in the art and can be employed in the context of the present invention to determine whether an individual has an elevated level of P-type IPGs, and so has or is at risk from pre-eclampsia, or has an elevated level of P-type IPGs consistent with the onset of labour. The purpose of such analysis may be used for diagnosis or prognosis to assist a physician in determining the severity or likely course of the pre-eclampsia and/or to optimise treatment of it, or to have warning of the onset of labour to clinically manage the birth. Examples of diagnostic methods are described in the experimental section below. Assays devices, kits and methods for the determination of the level or amount of IPGs in a sample are described herein and also in WO98/10791.

Preferred diagnostic methods rely on the detection of P-type IPGs, an elevated level of which was found to be associated with pre-eclampsia. The methods can employ biological samples such as blood, serum, tissue samples (especially placenta), or urine. Depending on the sample, it may be advantageous to carry out a pretreatment step, e.g. to remove cellular debris or unwanted contaminants from the sample.

In some embodiments, the present invention relies on the determination of one or more biological activities of P-type IPGs to assess whether the IPG is present at an elevated level in a biological sample. Alternatively or additionally, the concentration or amount of P-type IPGs in a sample may be determined.

The assay methods for determining the concentration of P-type IPGs typically employ a binding agent having binding sites capable of specifically binding to one or more of the P-type IPGs in preference to other molecules. Examples of binding agents include antibodies, receptors and other molecules capable of specifically binding P-type IPGs. Conveniently, the binding agent is immobilised on solid support, e.g. at a defined location, to make it easy to manipulate during the assay.

The sample is generally contacted with a binding agent under appropriate conditions so that P-type IPGs present in the sample can bind to the binding agent. The fractional occupancy of the binding sites of the binding agent can then be determined using a developing agent or agents. Typically, the developing agents are labelled (e.g. with radioactive, fluorescent or enzyme labels) so that they can be detected using techniques well known in the art. Thus, radioactive labels can be detected using a scintillation counter or other radiation counting device, fluorescent labels using a laser and confocal microscope, and enzyme labels by the action of an enzyme label on a substrate, typically to produce a colour change. The developing agent can be used in a competitive method in which the developing agent competes with the analyte (P-type IPG) for occupied binding sites of the binding agent, or non-competitive method, in which the labelled developing agent binds analyte bound by the binding agent or to occupied binding sites. Both methods provide an indication of the number of the binding sites occupied by the analyte, and hence the concentration of the analyte in the sample, e.g. by comparison with standards obtained using samples containing known concentrations of the analyte.

In the case of determinations of the amount of P-type IPGs in the sample (rather than its activity), the fractional occupancy of the binding sites of the binding agent can then be determined using a developing agent or agents. The developing agent can be used in a competitive method in which the developing agent competes with the analyte for occupied binding sites of the binding agent (e.g. using a labelled analogue of the analyte), or non-competitive method, in which the labelled developing agent binds analyte bound by the binding agent or to occupied binding sites (e.g. using an antibody with appropriate binding specificity). Both methods provide an indication of the number of the binding sites occupied by the analyte, and hence the concentration of the analyte in the sample, e.g. by comparison with standards obtained using samples containing known concentrations of the analyte.

### EXAMPLE 1

Gelatin Binds IPG Analytes

The first indication that gelatin was capable of binding IPG analytes was provided in the development of a sandwich assay for pre-eclampsia which employed a monoclonal capture antibody 2D1. The initial design of assay reported herein employed gelatin as a blocking agent for the solid support. Experiments to minimise the reagents for the assay employing three pre-eclamptic urines and one control pregnant urine were assayed with the 2Dl capture monoclonal antibody at three different coating levels, 2.5, 1.0 and 0.4 μg/ml. These results are shown in FIG. 1 and it can be seen that the activity of the four test samples was independent of the concentration of the capture monoclonal.

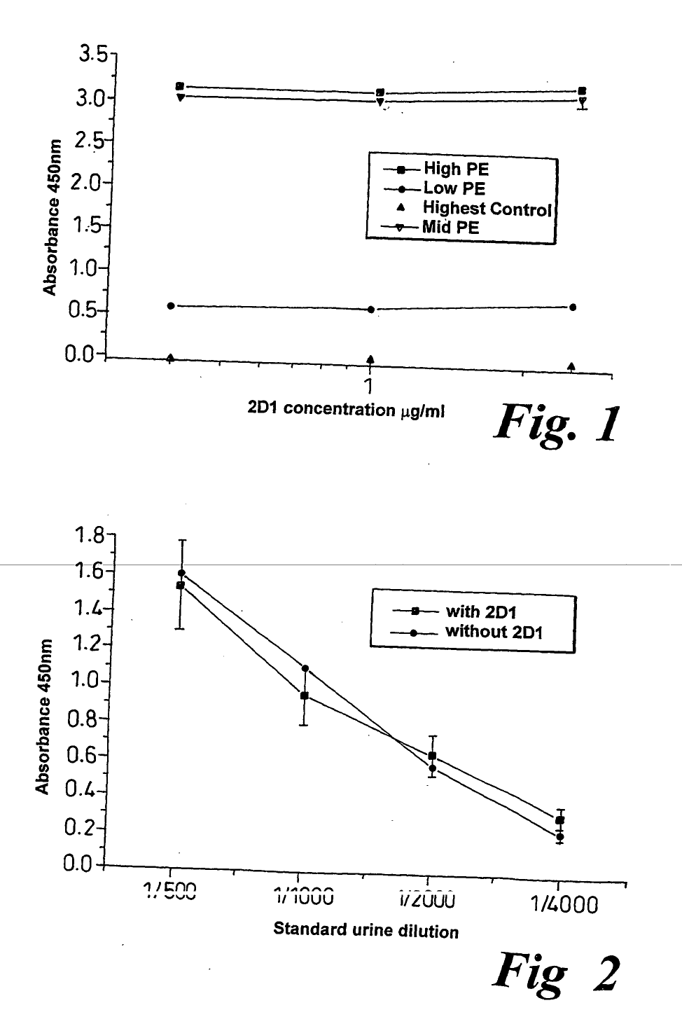

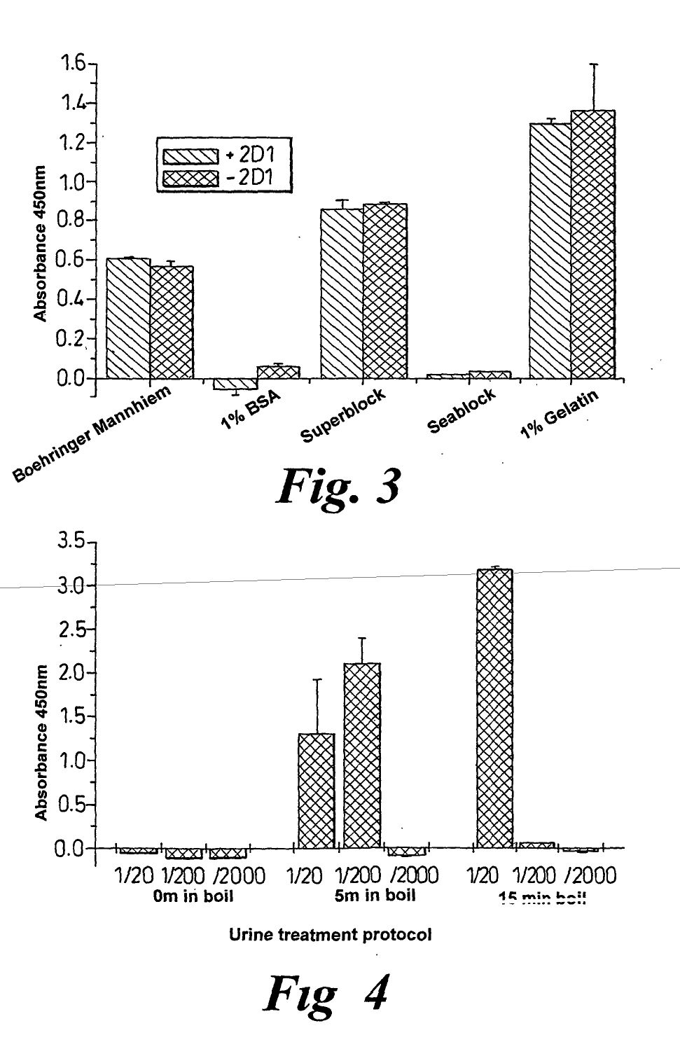

In a follow up experiment, the 2D1 capture agent was omitted and the data compared to that obtained when 2D1 was coated at 2.5 μg/ml. FIG. 2 shows that at a variety of pre-eclamptic urinary antigen concentrations, the activity was independent of the presence of the capture monoclonal.

In view of these surprising results, the ability of different blocking agents to bind IPG analytes such as the pre-eclamptic urinary antigen was investigated. Five commonly used blocking reagents were tested: Boehringer Mannheim's proprietary based gelatin blocker used in the assay shown in FIGS. 1 and 2, Pierce SEABLOCK®, the precise composition of which is a trade secret but is known to contain fish proteins, Pierce Super Block which contains gelatin, at least in part, 1% bovine serum albumin in phosphate buffered saline and 1% Sigma gelatin hydrolysate in phosphate buffered saline.

The data for these five blockers with and without the 2D1 capture monoclonal antibody is shown in FIG. 3. While the BSA and SEABLOCK® blockers failed to bind the pre-eclamptic urinary antigen, the three gelatin blockers bound the IPG analyte. Thus, this data clearly shows that a range of different gelatin based agents are able to capture the pre-eclamptic urinary antigen.

### EXAMPLE 2

Enzyme Linked Immunosorbent Assay of Pre-eclamptic Urinary Antigen Using Gelatin Based Capture and Polyclonal Antibody Detection

An ELISA assay was developed employing gelatin as the capture agent and rabbit polyclonal anti-IPG sera and a goat anti-rabbit horseradish peroxidase conjugated antibody as a two component developing agent. In this assay, 200 μl of 1% hydrolysed gelatin in PBS was added as blocking and capture reagent to the inner 6×10 arid of wells of a Maxisorp plate and incubated for 20 minutes at 37° C. in a sealed container in a water bath. The blocking reagent was then removed and the plate blotted dry by tapping it on tissue paper.

The pre-eclamptic urine samples and the controls were then diluted 1:100 in blocking reagent and 50 μl of dilute sample added to each well. The plate was then incubated for 40 minutes at 37° C. as above. After incubation, the wells were emptied and washed five times with 100 μl of 0.05% Tween 20/PBS. Next, 50 μl polyclonal anti-IPG rabbit sera diluted 1/10,000 in blocking reagent was added to each well and incubated for 30 minutes at 37° C., and then the wells were washed as above. The results of the assay were then obtained by adding 50 μl of 1 mg/ml goat anti-rabbit IgG horseradish peroxidase conjugated antibody, diluted 1/6000 in blocking reagent, followed by incubation for 20 minutes at 37° C. After a further washing step as above, 50 μl tetramethylbenzidine (TMB) solution (pre-warmed to 20° C. in incubator) was added to each well and incubated for 10 minutes at ambient temperature. The colour reaction was stopped by the addition of 50 μl of 1 M HCl per well, giving a blue to yellow colour change. The absorbance in each well was then read at 450 nm using a Molecular Devices 96 well plate reader.

### EXAMPLE 3

Pretreatment to Optimise the Assay

In order to improve the binding of the IPG analyte to the gelatin, pretreatment of the pre-eclamptic urinary antigen sample was investigated.

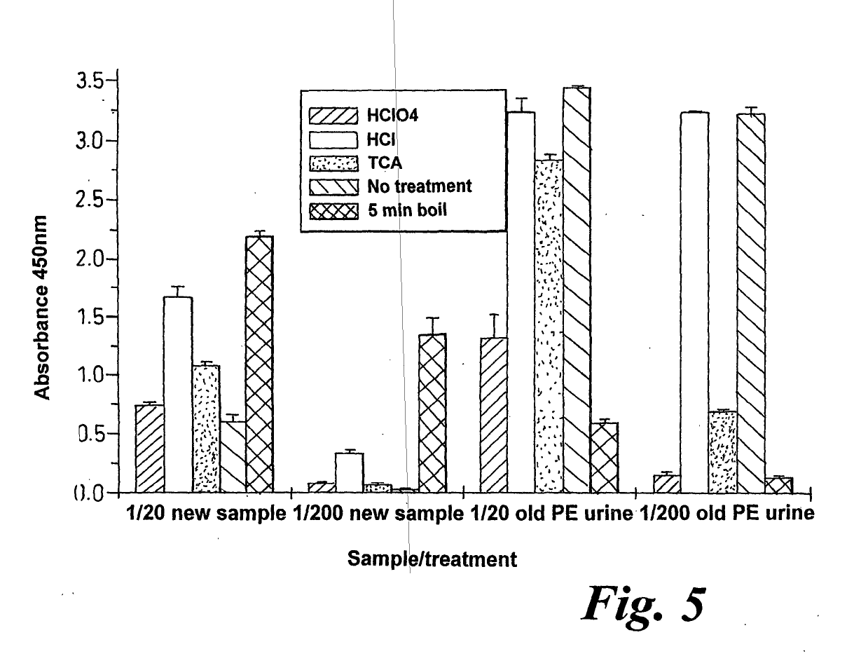

FIG. 5 shows the effect on the results of ELISA assays (carried out as in example 2) after a fresh pre-eclamptic urine sample had been placed in a boiling water bath for either 0, 5 or 15 minutes, at three different dilutions.

It can be seen that as little as 5 minutes heat treatment provided a large increase in the activity determined in the ELISA assay.

Table 1 below further shows comparative ELISA absorbance of urine sample PE/017 stored at either a nominal 4° C., 20° C. or −84° C. for 21 days with and without heat treatment (5 min, 90° C.).

Statistical analyses using Student's ‘t’ test compares aliquots of the same samples undergoing heat treatment, compared with the non-heat treated aliquots stored originally at the same temperature. The data shows that the urine reactivity increases slightly on storage at −84° C. but that there is a considerably greater increase on storage at −20° C. In addition all storage samples showed huge increases upon heat treatment of 5 minutes at 90° C.

TABLE 1

| | | | Heated at | | Negative |
|---|---|---|---|---|---|
| | Storage | Untreated | 90° C. for | Positive | control |
| | tem- | (OD | 5 min (OD | control | (OD |
| Sample | pera- | 450 nm) | 450 nm) | (OD 450 nm) | 450 nm) |
| number | ture | 1/20 | 1/20 | 1/500 | 1/20 |
| P.E/107 | 4° C. | 0.026 ± | 1.704 ± | 0.648 ± | 0.037 ± |
| | sterile | 0.007 | 0.084 | 0.039 | 0.028 |
| | | | p = | | |
| | | | 0.0008 | | |
| P.E/017 | −20° C. | 0.405 ± | 1.762 ± | | |
| | | 0.012 | 0.027 | | |
| | | | p < | | |
| | | | 0.0001 | | |
| P.E/017 | −84° C. | 0.076 ± | 1.353 ± | | |
| | | 0.007 | 0.051 | | |
| | | | p = | | |
| | | | 0.0004 | | |

FIG. 5 shows the effect of acid treatment, showing that HCl in particular was a good alternative to heat.

Table 2 shows a comparison of the absorbance of urine samples PE/011, PE/012, PE/013 and PE/014, frozen in liquid nitrogen and stored at either a nominal −20° C. or −84° C. for 21 days. Statistical analyses compare the reactivity determined by ELISA in aliquots of the same samples stored at different temperatures for the duration of the experiment. This data shows that four urine samples all show greater reactivity after prolonged storage at −20° C.

In addition to the use of high or low temperature to improve reactivity with gelatin capture agents, the use of chemical pretreatment was investigated. Accordingly, samples were treated with an equal volume of acid such that the final concentration was 0.25M HCl or perchloric acid or 10% TCA, left for 5 minutes at room temperature, spun to remove any precipitate and adjusted then pH to 7.0 with 1M trizma pH 8.0, before being diluted in blocking reagent and assayed. Two urine samples were tested, a relatively new PE sample and one over 3 years old.

TABLE 2

| | Samples | Samples | Positive | Negative |
|---|---|---|---|---|
| | stored at | stored at | control | control |
| Sample | −80° C. (OD | −20° C. (OD at | (OD at | (OD at |
| numbers | at 450 nm) | 450 nm) | 450 nm) | 450 nm) |
| dilution | 1.2 | 1.2 | | |
| P.E/011 | 0.044 ± | 0.292 ± 0.06 | Dilution | Dilution |
| | 0.029 | p = 0.008 | 1/500 | 1/20 |
| P.E/012 | 0.070 ± | 1.559 ± 0.332 | 3.850 ± | 0.000 ± |
| | 0.011 | p = 0.016 | 0.000 | 0.000 |
| P.E/013 | 0.029 ± | 1.109 ± 0.05 | Dilution | |
| | 0.037 | P < 0.0001 | 1.1000 | |
| P.E/014 | 0.000 ± | 0.586 ± 0.119 | 2.548 ± | |
| | 0.000 | p = 0.013 | 0.087 | |

The effect of pH adjustment using HCl was then investigated. The urine used in this study came from a clinically diagnosed pre-eclamptic patient and had been stored at 4° C. since collection. It had no diagnostic activity until it had been subjected to temperature or pH treatment.

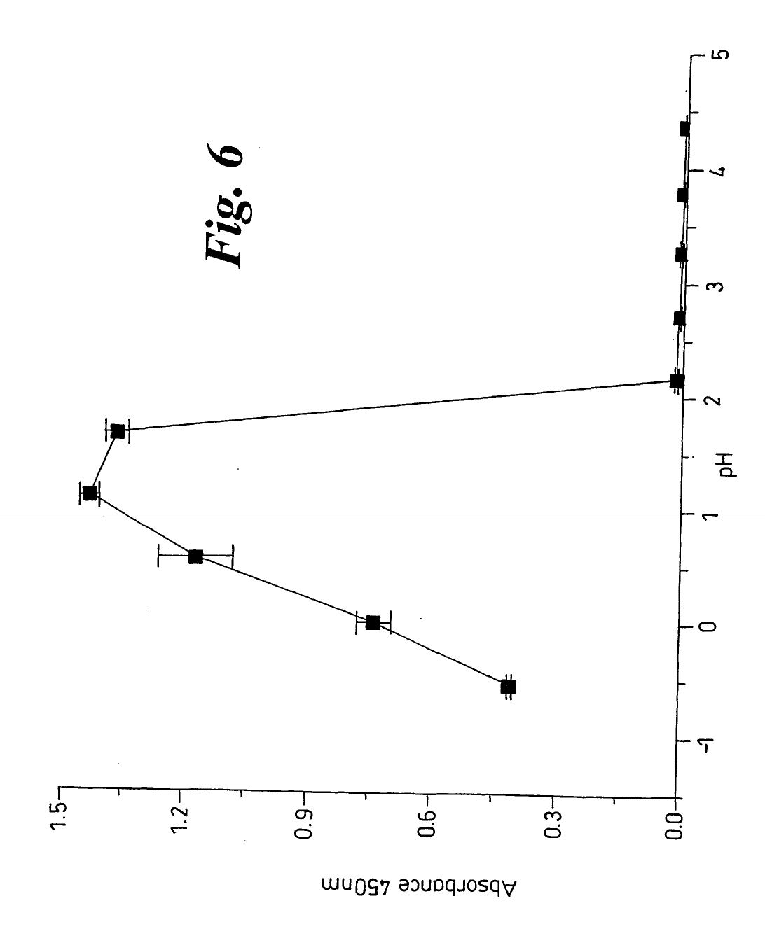

In the experiments, 7M stock HCl was diluted to provide a series of HCl standards from 7M to less than 0.1 mM. 10 μl urine samples were then add to 10 μl samples from the series of HCl standards and left in a capped Eppendorf tubes for 10 minutes. After incubation, the samples were neutralised by the addition of 1M trisma (pH8 5) and diluted to a final volume of 1 ml. These samples were then added to pre-coated gelatin plates prepared as described above and the reactivity of the analyte in the sample for the plate was determined. The results of these assays are shown in FIG. 6 which demontrates that the maximum reactivity was obtained using a pre-treatment pH of 1.25, and that pHs of less than pH 2 were sufficient in this system to produce good results.

While the above examples demonstrate the use of gelatin as a capture agent in an assay for the diagnosis of pre-eclampsia, those skilled in the art will be capable of adapting the assays described herein so that they can be used to determine the presence or amount other IPG analytes that are markers for pre-eclampsia or other conditions.

### EXAMPLE 4

A Phase I clinical trial was carried out to validate the diagnostic assay described herein for the diagnosis and prognosis of pre-eclampsia. The objectives of the trial were to confirm that pre-eclamptics could be identified using the assay, to examine the signal distribution at different stages of a pregnancy, to examine how early pre-eclamptics can be identified using the assay and to challenge the test with high risk and diabetic patients. The trial involved a total of 1024 patients and the important conclusions from the trial are reported below.

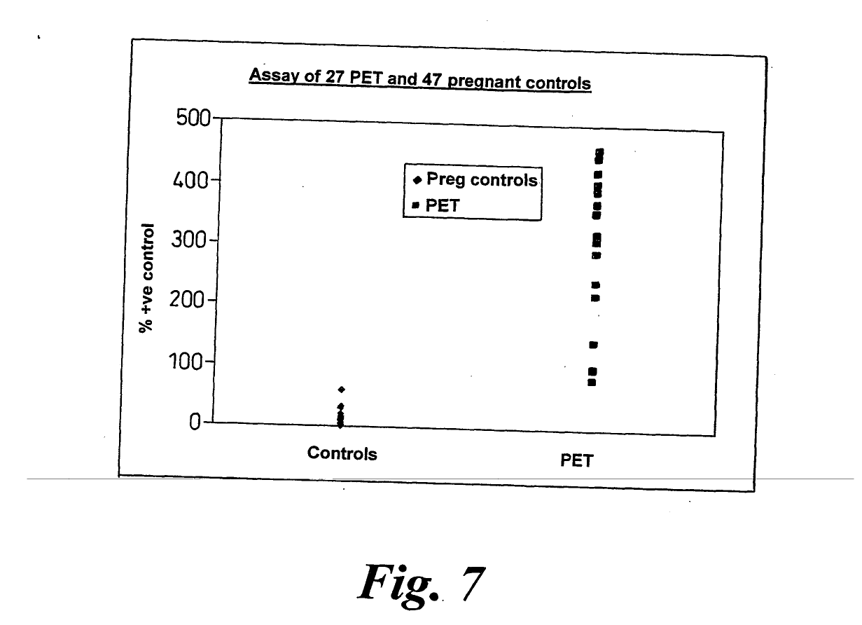

An initial study using samples from clinically diagnosed pre-eclamptic patients and non-pre-eclamptic control patients. The results of these assays are shown in FIG. 7 which depicts results from control pregnant subjects (n=47) and pre-eclamptic subjects (n=27). This clearly shows that the assays correctly identified all of the samples tested.

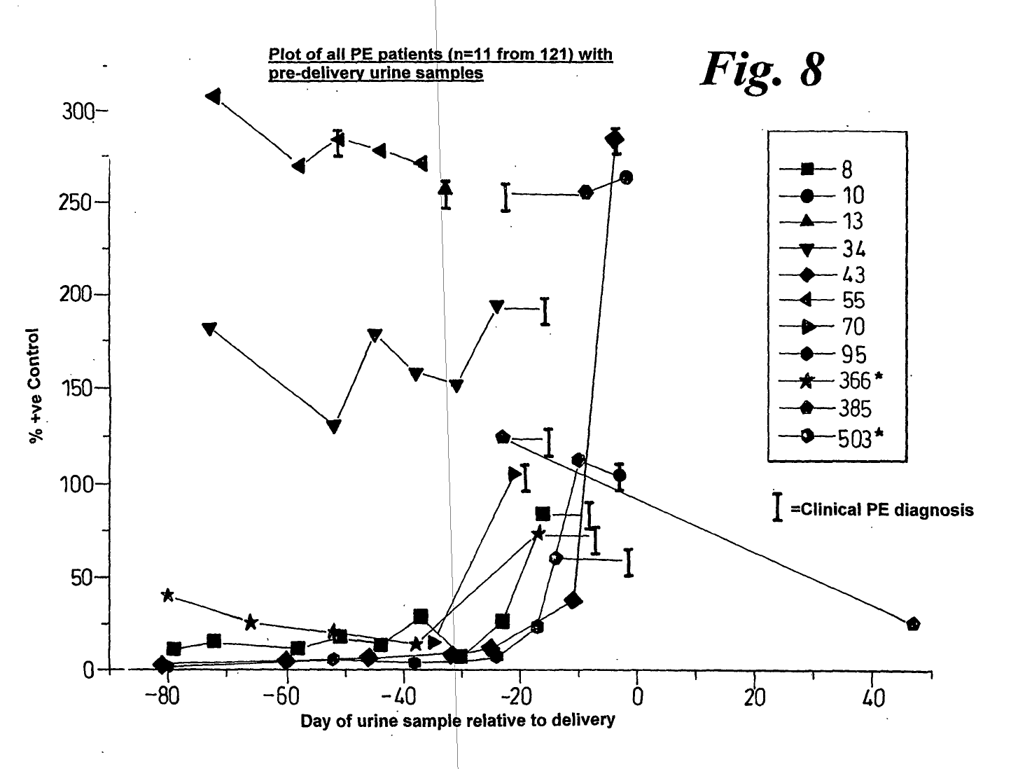

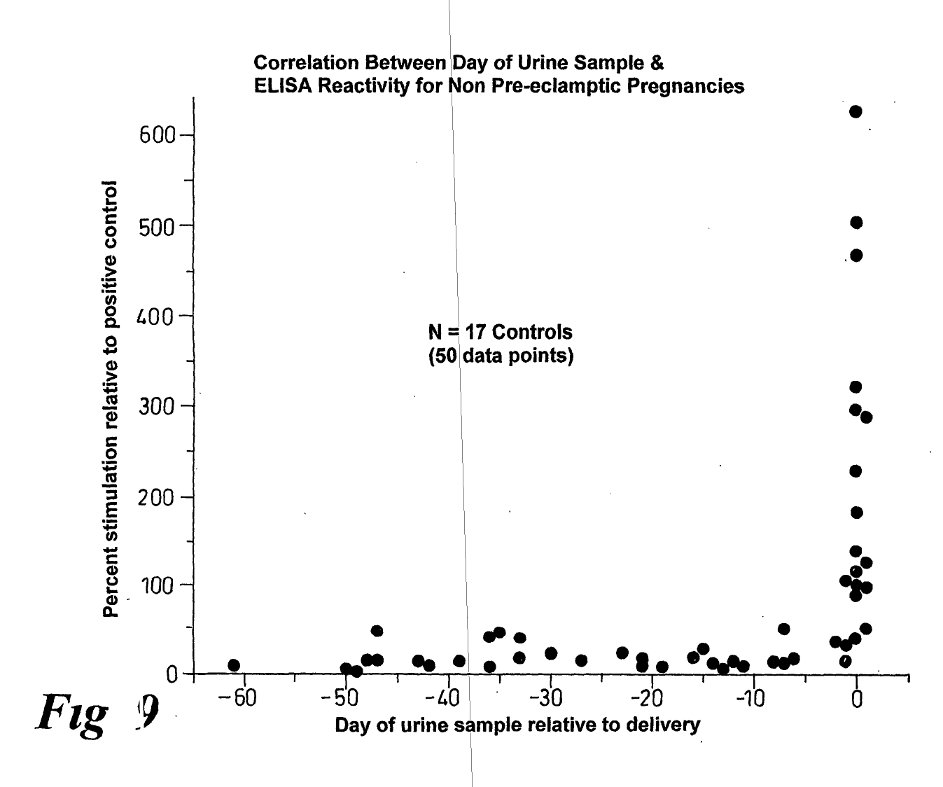

The signal development from the assay over time was then examined and exemplary results are shown in FIG. 8. This revealed that there are two distinct classes of pre-eclamptic patient that can be distinguished using the assay. The first type, represented by the graph for patients 55, 10 and 34, shows that the assay results were predictive of the development of pre-eclampsia about 2-4 weeks before the development of clinical symptoms in week 37. For this class of patients, the test is particularly useful as the early diagnosis of pre-eclampsia provides physicians with the opportunity to closely monitor, and if necessary treat, pre-eclampsia prior to the development of clinical symptoms. A second class of patients are represented by the example of patients 70, 366, 8, 95 and 43. In such patients, positive results from the assay are followed more quickly by the diagnosis of pre-eclampsia, i.e. the results of the test are diagnostic rather than being predictive some weeks earlier than the development of clinical symptoms. However, in both cases the assay is valuable as pre-eclampsia can be difficult to diagnose conventionally, and as the signal development patterns of both groups of patients are distinct. The study also demonstrated that the signal from the assay correlated to increasing severity of clinical symptoms.

A study was then carried out to determine whether type I diabetes in early pregnancy affects the outcome of the assay. This study used one or two samples per patient taken during the first trimeter and showed that type I diabetes and its treatment does not effect the outcome of the assay. A study examining the effect of type II diabetic patients showed that the assay still provided results although there was a higher background signal.

### EXAMPLE 5

The temporal relationship between the urine level of P-type IPGs in 50 patients at different times prior to delivery was investigated. FIG. 8 illustrates the dramatic rise in the value of P-type IPGs at labour relative to pre-labour values. This confirms that IPG levels can be used to determine the onset of labour. It may also be possible to use this marker to distinguish pre-term and normal term labour.

### EXAMPLE 6

The urinary levels of P-type IPGs was investigated in normal pregnant women in the 3rd trimester and during labour in a major London teaching hospital Obstetric unit. Mid-stream urine samples were obtained from 18 women in the third trimester of pregnancy (mean gestation 36.2 weeks). Serial urine samples were then obtained upon admission to the labour ward. Two patients were excluded due to the development of pregnancy-induced hypertension subsequent to recruitment. 4 patients were lost to follow-up, due to delivery at other units. All samples were frozen until analysis. All samples were assayed using a polyclonal ELISA for P-type IPGs. Samples were also assayed for protein content and creatinine content. The results of the polyclonal ELISA were expressed per mmol of creatinine. Paired t-test analysis was used between the two groups.

Paired recruitment (control) and labour samples were obtained for 12 women. 10 women laboured spontaneously, 2 women had induction of labour. The levels of P-type IPG were significantly higher in the labouring group (mean 12.6) compared to the non-labouring group (mean 2.7) p=0.01. Thus, the levels of P-type IPGs measured in an assay can be used to predict the onset of labour in patients.

Deposits

The deposit of hybridomas 2F7, 2D1 and 5H6 in support of this application was made at the European Collection of Cell Cultures (ECACC) under the Budapest Treaty by Rademacher Group Limited (RGL), The Windeyer Building, 46 Cleveland Street, London W1P 6DB, UK. The deposits have been accorded accession numbers:

| | | |
|---|---|---|
| 2F7 | 98051201 | 12 May 1999 |
| 2D1 | 98031212 | 9 Mar. 1998 |
| 5H6 | 98030901 | 9 Mar. 1998 |

RGL give their unreserved and irrevocable consent to the materials being made available to the public in accordance with appropriate national laws governing the deposit of these materials, such as Rules 28 and 28a EPC. The expert solution under Rule 28(4) EPC is also hereby requested.

### REFERENCES

The references cited herein are all expressly incorporated by reference.

Caro et al, Biochem. Molec. Med., 61:214-228, 1997.

Kunjara et al, In: Biopolymers and Bioproducts: Structure, Function and Applications, Ed Svati et al, 301-305, 1995.

Rademacher et al, Brazilian J. Med. Biol. Res., 27:327-341, 1994.

WO98/01116 (Rademacher Group Limited).

WO98/01117 (Rademacher Group Limited).

WO98/10791 (Rademacher Group Limited).

WO98/11435 (Rademacher Group Limited).

WO99/47565(Rademacher Group Limited).

1. Use of gelatin as a binding agent in an assay for determining the presence or amount of an inositol phosphoglycan (IPG) analyte in a sample.

2. The use of claim 1, wherein the presence or amount of the IPG analyte bound to the binding agent is determined using a developing agent which is capable of binding to the IPG analyte.

3. The use of claim 1 or claim 2, wherein the developing agent comprises a detectable label, a moiety capable of being converted into a detectable label or is capable of specifically interacting with a further detectably labelled reagent.

4. The use of claim 3, wherein the label is an enzyme, a fluorescent compound, a chemiluminescent compound, a radioactive isotope, a coloured particle, a gold particle, a dye, or a magnetic particle.

5. The use of any one of the preceding claims, wherein the developing agent comprises an anti-IPG antibody.

6. The use of claim 5, wherein the developing agent is monoclonal antibody 2D1 or 5H6, deposited at ECACC under accession number 98031212 or 98030901 respectively.

7. The use of any one of the preceding claims, wherein the IPG analyte comprises a lipid group.

8. The use of any one of the preceding claims, wherein the presence or amount of the IPG analyte is for diagnosing pre-eclampsia.

9. The use of claim 8, wherein the assay is capable of predicting the onset of pre-eclampsia at least 2 weeks prior to clinical symptoms.

10. The use of any one of the preceding claims, wherein the gelatin is Boehringer Mannheim's proprietary gelatin blocker, Pierce Superblock and Sigma gelatin hydrolysate.

11. The use of any one of the preceding claims, wherein the sample is urine, blood, serum, plasma, saliva, tears or mucus sample.

12. The use of claim 11, wherein the sample is a urine sample.

13. The use of any one of the preceding claims, wherein the sample is treated to cause the IPG analyte to form micelles.

14. The use of claim 13, wherein the treatment step comprises freezing at about −20° C., heating to about 100° C. or chemical treatment.

15. The use of claim 14, wherein the chemical treatment comprises contacting the sample with an acid.

16. The use of claim 15, wherein the acid is hydrochloric acid.

17. A method of diagnosing a condition associated with the presence or amount of an inositol phosphoglycan (IPG) analyte in a sample from a patient, the method comprising:
contacting the sample with a solid support having a capture zone comprising gelatin which is capable of binding the IPG analyte present in the sample;
contacting the solid support with a developing agent capable of binding to the captured IPG analyte; and,
detecting the developing agent to determine the presence or amount of the IPG analyte in the sample.

18. The method of claim 17, wherein the condition is pre-eclampsia.

19. The method of claim 17 or claim 18, wherein the IPG analyte comprises a lipid group.

20. The method of any one of claims 17 to 19, wherein the solid support is a lateral flow assay device.

21. The method of any one of claims 17 to 20, wherein the gelatin is Boehringer Mannheim's proprietary gelatin blocker, Pierce Superblock and Sigma gelatin hydrolysate.

22. The method of any one of claims 17 to 21, wherein the gelatin is coated on the solid support.

23. The method of any one of claims 17 to 22, wherein the developing agent comprises a detectable label, a moiety capable of being converted into a detectable label or is capable of specifically interacting with a further detectably labelled reagent.

24. The method of claim 23, wherein the label is an enzyme, a fluorescent compound, a chemiluminescent compound, a radioactive isotope, a coloured particle, a gold particle, a dye, or a magnetic particle.

25. The method of any one of claims 17 to 24, wherein the developing agent is an anti-IPG antibody.

26. The method of claim 25, wherein the developing agent is a monoclonal antibody 2D1 or 5H6 deposited at ECACC under accession number 98031212 or 98030901 respectively.

27. The method of any one of claims 17 to 26, wherein the sample is urine, blood, serum, plasma, saliva, tears or mucus sample.

28. The method of claim 27, wherein the sample is a urine sample.

29. The method of any one of claims 17 to 28, further comprising pre-treating the sample so that the IPG analyte forms micelles.

30. The method of claim 29, wherein the pre-treating step comprises freezing at about −20° C., heating to about 100° C. or chemical treatment.

31. The method of claim 30, wherein the chemical treatment comprises contacting the sample with an acid.

32. The method of claim 31, wherein the acid is hydrochloric acid.

33. A kit for diagnosing a condition associated with the presence or amount of an inositol phosphoglycan (IPG) analyte in a sample from a patient, the kit comprising:
a solid support having a capture zone comprising gelatin which is capable of binding to the IPG analyte present in the sample; and,
a developing agent capable of binding to the IPG analyte bound to the capture zone, the developing agent comprises a detectable label, a moiety capable of being converted into a detectable label or is capable of specifically interacting with a further detectably labelled reagent.

34. The kit of claim 33, wherein the presence of amount of the IPG analyte is for diagnosing pre-eclampsia.

35. The kit of claim 34, wherein the kit is capable of diagnosing pre-eclampsia at least 2 weeks prior to the manifestation of clinical symptoms.

36. The kit of any one of claims 33 to 35, wherein the developing agent is an anti-IPG antibody.

37. The kit of any one of claims 33 to 36, wherein the IPG analyte comprises a lipid group.

38. The kit of any one of the claims 33 to 37, wherein the gelatin is Boehringer Mannheim's proprietary gelatin blocker, Pierce Superblock and Sigma gelatin hydrolysate.

39. The kit of any one of claims 33 to 38 wherein the gelatin is coated on the solid support.

40. A lateral flow device for determining the presence or amount of an inositol phosphoglycan (IPG) analyte in a sample, the device comprising a solid support comprising in sample flow order:
(a) a sample addition zone;
(b) a pretreatment zone for reacting with the sample;
(c) a capture zone comprising gelatin which is capable of binding to the IPG analyte present in the sample;
wherein the presence of amount of the IPG analyte is determined using a developing agent capable of binding to the IPG analyte bound to the capture zone, the developing agent comprises a detectable label, a moiety capable of being converted into a detectable label or is capable of specifically interacting with a further detectably labelled reagent.

41. The assay device of claim 40, wherein the presence of amount of the IPG analyte is for diagnosing pre-eclampsia.

42. The assay device of claim 40 or claim 41, wherein the developing agent is an anti-IPG antibody.

43. The assay device of any one of claims 40 to 42, wherein the IPG analyte comprises a lipid group.

44. The assay device of any one of the claims 40 to 43, wherein the gelatin is Boehringer Mannheim's proprietary gelatin blocker, Pierce Superblock and Sigma gelatin hydrolysate.

45. The assay device of any one of claims 40 to 44, wherein the gelatin is coated on the solid support.

46. The assay device of any one of claims 40 to 45, wherein the solid support is formed from a cellulose ester material.

47. The assay device of any one of claims 40 to 46, wherein the pre-treatment zone adjusts the pH of the sample to enhance the binding of the IPG analyte to the gelatin capture phase.

48. A method of predicting the onset of labour in a mammal, the method comprising determining the amount of activity of P-type IPGs in a sample obtained from the mammal.

49. The method of claim 48, wherein the method distinguishes pre-term labour from normal term labour.

50. The method of claim 48 or claim 49, wherein the mammal is a human patient.

51. The method of claim 48 or claim 49, wherein the mammal is ovine or bovine.

52. The method of any one of claims 48 to 51, wherein the activity of the P-type IPGs is determined using an assay for a P-type IPG biological activity.

53. The method of any one of claims 48 to 52, wherein the level of the P-type IPGs is determined in an assay measuring activation of pyruvate dehydrogenase phosphatase by P-type IPGs.

54. The method of any one of claims 48 to 53, wherein the level of the P-type IPGs is determined using a binding agent capable of specifically binding P-type IPGs.

55. The method of claim 54, the method comprising the steps of:
(a) contacting a biological sample obtained from the patient with a solid support having immobilised thereon binding agent having binding sites specific for one or more P-type IPGs;
(b) contacting the solid support with a labelled developing agent capable of binding to unoccupied binding sites, bound P-type IPGs or occupied binding sites; and,
(c) detecting the label of the developing agent specifically binding in step (b) to obtain a value representative of the amount of the P-type IPGs in the sample.

56. The method of claim 55, the method comprising the further step of:
(d) correlating the value obtained in step (c) with levels of P-type IPGs in control subjects to determine whether the patient has an elevated level of P-type IPGs.

57. The method of claim 55 or claim 56, wherein the binding agent is an anti-P-type IPG antibody.

58. The method of any one of claims 55 to 57, wherein an elevated level of the P-type IPGs is greater than about 2 times the level in control subjects.

59. The method of any one of claims 48 to 58, wherein the sample is a blood, serum, tissue or urine sample.
